(19) United States
(12) Patent Application Publication
(10) Pub. No.: US 2006/0141076 A1
(43) Pub. Date: Jun. 29, 2006

(54) Morinda citrifolia based compositions and methods for inhibiting xanthine oxidase

(76) Inventors: Afa Kehaati Palu, American Fork, UT (US); Chen Su, West Jordan, UT (US); Bing-Nan Zhou, Pleasant Grove, UT (US); Claude Jarakae Jensen, Cedar Hills, UT (US); Kim Asay, Orem, UT (US); Stephen P. Story, Alpine, UT (US)

Correspondence Address: KIRTON AND MCCONKIE;1800 EAGLE GATE TOWER, 60 EAST SOUTH TEMPLE, P O BOX 45120, SALT LAKE CITY, UT, 84145-0120, US

(21) Appl. No.: 11/257,681

(22) Filed: Oct. 25, 2005

Related U.S. Application Data
(60) Provisional application No. 60/624,101, filed on Nov. 1, 2004.

(51) Int. Cl. A61K 36/45; A61K 36/73; A61K 36/746

(52) U.S. Cl. 424/732; 424/769; 424/765

(57) ABSTRACT

The present invention relates to the inhibition of xanthine oxidase. In particular, the present invention relates to systems and methods for utilizing Morinda citrifolia L. based compositions and methods to inhibit xanthine oxidase activity.

### RELATED APPLICATIONS

This application is claims priority to U.S. Provisional Patent Application Ser. No. 60/624,101, filed Nov. 1, 2004.

### BACKGROUND OF THE INVENTION

1. Field of the Invention

The present invention relates to the inhibition of xanthine oxidase. In particular, the present invention relates to systems and methods for utilizing Morinda citrifolia L. based compositions and methods to inhibit xanthine oxidase activity.

2. Background and Related Art

Many diseases or symptoms of diseases arise from a deficiency or excess of a specific metabolite in the body. Xanthine oxidase (XO) is a flavoprotein enzyme, found in most species (from bacteria to human). In general XO catalyses the oxidative hydroxylation of purine substrates and subsequent reduction of O₂ at the flavin center with generation of reactive oxygen species (ROS), either superxoide anion radical or hydrogen peroxide. One of the specific reactions in which XO participates is the oxidation of hypoxanthine and the oxidation of xanthine. XO catalyses the oxidation of hypoxanthine to xanthine and then to uric acid. Uric acid plays an important role in gout. Inhibition of XO decreases the uric acid levels, and results in an antihyperuricemic effect. Allopurinol is prescribed as a xanthine oxidase inhibitor used in the treatment of gout and gout-like diseases. XO serum levels are significantly increased in various pathological states like hepatitis, inflammation, ischemia reperfusion, carcinogenesis and aging and the ROS generated in the enzymatic process are involved in oxidative damage. It is therefore possible that the inhibition of this enzymatic pathway would be therapeutically beneficial.

### SUMMARY AND OBJECTS OF THE INVENTION

The present invention relates to the inhibition of xanthine oxidase. In particular, the present invention relates to systems and methods for utilizing Morinda citrifolia based compositions and methods to inhibit xanthine oxidase activity to ameliorate gout and gout-like diseases.

In some embodiments Morinda citrofolia juice, when taken in sufficient amounts, is a useful xanthine oxidase inhibitor. In some embodiments the inhibition of enzyme function results in a decrease in the uric acid levels resulting in an antihyperuricemic effect.

In some embodiments the consumption of Morinda citrifolia based products is useful in treating diseases caused by an excess of uric acid such as gout. In some embodiments Gout can be treated by consuming between three ounces and one cup twice daily depending on body weight and concentration of the Morinda citrifolia product.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates the inhibition curve for Morinda citrifolia;

FIG. 2 illustrates the inhibition curve for Allopurinol.

### DETAILED DESCRIPTION OF THE INVENTION

The present invention relates to the inhibition of xanthine oxidase. In particular, the present invention relates to systems and methods for utilizing Morinda citrifolia based compositions and methods to inhibit xanthine oxidase activity to ameliorate gout and gout-like diseases.

In the disclosure and in the claims the singular forms “a,” “an,” and “the” include plural referents unless the context clearly dictates otherwise.

In describing and claiming the present disclosure, the following terminology will be used in accordance with the definitions set out below. As used herein, the terms “comprising,” “including,” “containing,” “characterized by,” and grammatical equivalents thereof are inclusive or open-ended terms that do not exclude additional, unrecited elements or method steps. As used herein, the phrase “consisting of” and grammatical equivalents thereof exclude any element, step, or ingredient not specified in the claim. As used herein, an “effective amount” is an amount sufficient to effect beneficial or desired results. An effective amount can be administered in one or more administrations, applications or dosages. For example, an effective amount of a Morinda citrifolia based composition is an amount sufficient to inhibit xanthine oxidase, inhibit the production of uric acid, and prevent/ameliorate conditions related to gout.

The following disclosure of the present invention is grouped into three subheadings, namely “General Discussion of Morinda citrifolia and the Methods Used to Produce Processed Morinda citrifolia Products,” “Formulations and Methods of Administration” and “Inhibiting Xanthine Oxidase.” The utilization of the subheadings is for convenience of the reader only and is not to be construed as limiting in any sense.

1. General Discussion of Morinda citrifolia and the Methods Used to Produce Processed Morinda citrifolia Products

The Indian Mulberry or Noni plant, known scientifically as Morinda Citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in the nutraceutical used to inhibit xanthine oxidase. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 120 days. Most fruit containers are held for 7 to 14 days before processing. The containers can optionally be stored under refrigerated conditions or ambient/room temperature conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a dietary fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid. The present invention contemplates utilizing all parts of the M. citrifolia plant alone, in combination with each other or in combination with other ingredients. The above listed portions of the M. citrifolia plant is not an exhaustive list of parts of the plant to be used but are merely exemplary. Thus, while some of the parts of the M. citrifolia plant are not mentioned above (e.g., seed from the fruit, the pericarp of the fruit, the bark or the plant) the present invention contemplates the use of all of the parts of the plant.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

The compositions containing Morinda citrifolia may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients, which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

2. Formulations and Methods of Administration

The present invention provides nutraceutical formulations and methods for inhibiting xanthine oxidase with a Morinda citrifolia-based nutraceutical formulation without any significant tendency to cause side effects. The Morinda citrifolia is incorporated into various carriers or nutraceutical compositions suitable for in vivo treatment of a patient. For instance, the processed Morinda citrifolia may be ingested, introduced through an intravenous injection or feeding, or otherwise internalized as is appropriate and directed.

In one exemplary embodiment, the nutraceutical composition of the present invention comprises one or more of a processed Morinda citrifolia product present in an amount by weight between about 0.01 and 80 percent by weight. Several embodiment of formulations are provided below. However, these are only intended to be exemplary as one ordinarily skilled in the art will recognize other formulations or compositions comprising the processed Morinda citrifolia product.

The processed Morinda citrifolia product is an active ingredient or contains one or more active ingredients, such as Quercetin and Rutin, and others, for effectuating the prevention, inhibition xanthine oxidase. The effects of the processed Morinda citrifolia product are synergistically enhanced by the presence of other active ingredients. One embodiment of the present invention comprises a processed Morinda citrifolia product in formulation, which inhibits xanthine oxidase. Active ingredients may be extracted out of various parts of the Morinda citrifolia plants using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

The processed Morinda citrifolia product may be formulated with various other ingredients to produce various compositions, such as a nutraceutical composition, an internal composition, or others. The ingredients to be utilized in a nutraceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the nutraceutical composition will most likely be consumed orally, it may contain one or more agents selected from the group consisting of sweetening agents, flavoring agents, coloring agents, preserving agents, and other medicinal agents as directed.

The ingredients to be utilized in a topical dermal composition are also any that are safe for internalizing into the body of a mammal and may exist in various forms, such as gels, lotions, creams, ointments, etc., each comprising one or more carrier agents. The ingredients for systemically administered formulations may also comprise any known in the art.

In one exemplary embodiment, the present invention further features a method of administering a nutraceutical composition to a mammal for the inhibition of xanthine oxidase. The method comprises the steps of (a) formulating a nutraceutical composition comprising in part a processed Morinda citrifolia product present in an amount between about 0.01 and 80 percent by weight, wherein the composition also comprises a carrier, such as water or purified water, and other natural or artificial ingredients; (b) administering the nutraceutical composition into the body such that the processed Morinda citrifolia product is sufficiently internalized; (c) repeating the above steps as often as necessary to provide an effective amount of the processed Morinda citrifolia product.

The step of administering the nutraceutical composition into the body comprises ingesting the composition orally through one of several means. Specifically, the nutraceutical composition may be formulated as a liquid, gel, solid, or some other type that would allow the composition to be quickly and conveniently digested. Once sufficiently internalized, the administered nutraceutical composition may then begin to act to inhibit xanthine oxidase in the subject. In addition, the step of administering the nutraceutical composition may include injecting the composition into the body using an intravenous pump.

In one exemplary embodiment, the nutraceutical composition is administered by taking between two ounces, of the nutraceutical composition every two hours each day, or at least twice a day. The nutraceutical composition is to be taken on an empty stomach, meaning at a period of time at least two hours prior to consumption of any food or drink. Of course, one ordinarily skilled in the art will recognize that the amount of composition and frequency of use may vary from individual to individual. It is contemplated that a person may ingest less than one-half ounce, or more than ten ounces of the nutraceutical composition claimed in the present invention. Thus, the present invention contemplates the administration of one ounce, two ounces, three ounces or any volume of the formulations necessary to achieve the desired result including more than ten ounces per administration.

The following tables illustrate or represent some of the preferred formulations or compositions contemplated by the present invention. As stated, these are only intended as exemplary embodiments and are not to be construed as limiting in any way.

Formulation One

| Ingredients | Percent by Weight |
|---|---|
| Morinda citrifolia puree juice | 100% |
| or fruit juice | |

Formulation Two

| Ingredients | Percent by Weight |
|---|---|
| Morinda citrifolia fruit juice | 50-99.99% |
| water | 0.1-50% |

Formulation Three

| Ingredients | Percent by Weight |
|---|---|
| Morinda citrifolia fruit juice | 50-99.99% |
| non-Morinda citrifolia-based fruit juices | 0.1-50% |

Formulation Four

| Ingredients | Percent by Weight |
|---|---|
| Morinda citrifolia fruit juice | 50-90% |
| water | 0.1-50% |
| non-Morinda citrifolia-based fruit juices | 0.1-30% |

Formulation Five

| Ingredients | Percent by Weight |
|---|---|
| Morinda citrifolia puree juice | 50-99.9% |
| water | 0.1-50% |

Formulation Six

| Ingredients | Percent by Weight |
|---|---|
| Morinda citrifolia puree juice | 50-99.9% |
| non-Morinda citrifolia-based fruit juices | 0.1-50% |

Formulation Seven

| Ingredients | Percent by Weight |
|---|---|
| Morinda citrifolia puree juice | 50-90% |
| water | 0.1-50% |
| non-Morinda citrifolia-based fruit juices | 0.1-30% |

Formulation Eight

| Ingredients | Percent by Weight |
|---|---|
| Morinda citrifolia dietary fiber | 0.1-50% |
| water | 1-99.9% |
| non-Morinda citrifolia-based fruit juices | 1-99.9% |

Formulation Nine

| Ingredients | Percent by Weight |
|---|---|
| Morinda citrifolia dietary fiber | 0.1-50% |
| water | 1-99.9% |
| Morinda citrifolia fruit juice or puree juice | 1-99.9% |

Formulation Ten

| Ingredients | Percent by Weight |
|---|---|
| Morinda citrifolia oil | 0.1-50% |
| carrier medium | 70-99.9% |
| other ingredients | 1-95% |

Formulation Eleven

| Ingredients | Percent by Weight |
|---|---|
| Morinda citrifolia product | 10-80% |
| carrier medium | 20-90% |

Formulation Twelve

| Ingredients | Percent by Weight |
|---|---|
| Morinda citrifolia product | 5-80% |
| carrier medium | 20-95% |

Formulation Thirteen

| Ingredients | Percent by Weight |
|---|---|
| Morinda citrifolia oil or oil extract | 0.1-50% |
| carrier medium | 20-90% |

Formulation Fourteen

| Ingredients | Percent by Weight |
|---|---|
| Morinda citrifolia puree juice | 0.1-80% |
| and/or fruit Juice | |
| Morinda citrifolia oil | 0.1-50% |
| carrier medium | 20-90% |

Formulation Fifteen

| Ingredients | Percent by Weight |
|---|---|
| Morinda citrifolia puree juice concentrate | 100% |
| and/or fruit juice | |
| concentrate | |

Formulation Sixteen

| Ingredients | Percent by Weight |
|---|---|
| Morinda citrifolia fruit juice concentrate | 50-99.99% |
| and/or puree juice | |
| concentrate | |
| water | 0.1-50% |

Formulation Seventeen

| Ingredients | Percent by Weight |
|---|---|
| Morinda citrifolia Juice | 0.1-99.8% |
| Quercetin | 0.1-90% |
| Rutin | 0.1-90% |

In one preferred method, a person wanting to inhibit xanthine oxidase to treat gout as described above takes, or is administered, at least one ounce of Formulation One in the morning on an empty stomach, and at least one ounce at night on an empty stomach, just prior to retiring to bed. In one example, which is not meant to be limiting in any way, the beneficial Morinda Citrifolia is processed into Tahitian Noni juice manufactured by Morinda, Incorporated of Orem, Utah.

According to the present invention, these particular methods of introducing an internal composition may comprise any method of actually introducing the internal composition to the subject for the purpose of inhibiting xanthine oxidase. Although the particular methods are many, the present invention recognizes that the internal composition may be introduced intravenously, transdermally, orally, or systemically. No matter what method is employed, it is important to regulate the amount of active ingredient that the subject is exposed to so that the appropriate anti-cancer objectives are accomplished.

The carrier medium may comprise any ingredient capable of being introduced into the body of a mammal, and that is capable of providing the carrying medium to the processed Morinda citrifolia product. Specific carrier mediums formulations are well known in the art and are not described in detail herein. The purpose of the carrier medium is as stated, to provide a means to embody the processed Morinda citrifolia product within the internal composition that is capable of being introduced into the body of the subject to be treated.

3. Inhibiting Xanthine Oxidase

The present invention relates generally to the use of various Morinda citrifolia based compositions to inhibit xanthine oxidase enzymatic capabilities. In general the present invention contemplates utilizing various Morinda citrifolia based compositions and methods to inhibit xanthine oxidase's role as a catalyst in the oxidative hydroxylation of purine substrates and subsequent reduction of O₂ at the flavin center with generation of reactive oxygen species (ROS), either superxoide anion radical or hydrogen peroxide (the oxidative half-reaction).

More particularly, the present invention contemplates utilizing various Morinda citrifolia based compositions to inhibit, prevent or ameliorate a variety of diseases and maladies associated with xanthine oxidase activity. One of the specific reactions in which xanthine oxidase participates is the oxidation of hypoxanthine and the oxidation of xanthine. Xanthine oxidase catalyses the oxidation of hypoxanthine to xanthine and then to uric acid. Uric acid plays an important role in gout. Inhibition of xanthine oxidase decreases the uric acid levels, and results in an antihyperuricemic effect.

In addition to treating gout the present invention contemplates utilizing various Morinda citrifolia based compositions and methods to ameliorate or prevent a plethora of diseases. Xanthine oxidase serum levels are significantly increased in various pathological states like hepatitis, inflammation, ischemia reperfusion, carcinogenesis and aging and the ROS generated in the enzymatic process are involved in oxidative damage. It is therefore contemplated by the present invention that the inhibition of this enzymatic pathway would be beneficial in the treatment of a variety of diseases.

The following examples set forth and present the effects of Morinda citrifolia on xanthine oxidase. These examples are not intended to be limiting in any way, but are merely illustrative of the beneficial, advantageous, and remedial effects of Morinda citrifolia on xanthine oxidase, including preventing and treating gout. Other non-limiting examples of the present invention are described below.

### EXAMPLE ONE

In the present example, a patient with gout desires to treat the condition with a nonprescription, over-the-counter preparation. To treat gout, the individual consumes an identified prescribed amount of a food product composition containing processed Morinda citrifolia fruit juice. The person intermittently consumes the food product containing the processed Morinda citrifolia fruit juice until the conditions associated with gout are ameliorated.

In another embodiment of the present invention a person interested in preventing or inhibiting the development of gout may consume a food product compositions containing processed Morinda citrifolia fruit juice. The person intermittently consumes the food product containing the processed Morinda citrifolia fruit juice for an indefinite period to continually prevent or inhibit the development of gout, or conditions associated therewith.

### EXAMPLE TWO

Assays were performed which demonstrate that processed Morinda citrifolia products inhibit xanthine oxidase. Xanthine Oxidase was obtained from Bovine butter milk. 165 μM Xanthine was utilized as a stubstrate. The vehicle for the assays contained 1% DMSO. Samples were pre-incubated for thirty minutes at 37° C. Samples were incubated for thirty minutes at 25° C. The incubation buffer contained 33.3 mM phosphate and had a pH of 7.5. Uric Acid concentration was quantitated by spectrophotometer. Reference compound data was produced utilizing known Xanthine Oxidase inhibitor 2.4 and 3.29 μM Allopurinol.

Biochemical assay results are presented as the percent inhibition of specific binding or activity throughout this disclosure. All other results are expressed in terms of that assay's quantitation method. For primary assays, only the lowest concentration with a significant response judged by the assays' criteria, is shown in this summary. Where applicable, either the secondary assay results with the lowest dose, concentration meeting the significance criteria or, if inactive, the highest dose, concentration that did not meet the significance criteria is shown. Primary screening in duplicate with quantitative data (e.g., IC₅₀±SEM and nH) are shown where applicable for assays.

Methods employed in this study were adapted from the scientific literature to maximize reliability and reproducibility. Reference standards were run as an integral part of each assay to ensure the validity of the results obtained.

TABLE 1

| Target | Species | Conc. | % INH. |
|---|---|---|---|
| Xanthine Oxidase | bov | 1% | 184 |

As shown in Table 1, bovine xanthine oxidase was significantly inhibited when exposed to 1% processed Morinda citrifolia juice.

### EXAMPLE THREE

Additional assays were performed with 5% processed Morinda citrifolia juice. The protocols, materials and methods detailed in Example 2 above were utilized to produce the data in TABLE 2.

TABLE 2

| | | | % | |
|---|---|---|---|---|
| Target | SPECIES | Conc. | INH | IC₅₀ |
| Xanthine oxidase | bov | 5% | 113 | 3.80% |

As shown in Table 2, bovine xanthine oxidase was significantly inhibited when exposed to 5% processed Morinda Citrifolia juice. The results achieved in Example Two and Example Three were surprising, because the inhibitory effect of 1% and 5% processed Morinda citrifolia was more than other drugs marketed to inhibit xanthine oxidase. For example, Allopurinol produces only approximately 90 percent inhibition of xanthine oxidase.

### EXAMPLE FOUR

Additional assays were performed, which demonstrate the relationship percent inhibition of xanthine oxidase and concentration of processed Morinda citrifolia. The protocols described previously were utilized to produce the following data.

TABLE 3

| | | | % | |
|---|---|---|---|---|
| Target | SPECIES | Conc. | INH | IC₅₀ |
| Xanthine oxidase | bov | 5% | 77 | 3.04% |

TABLE 4

| Target | Spp | n= | CONC. | % Inhibition | IC₅₀ |
|---|---|---|---|---|---|
| Xanthine | bov | 2 | 10.00% | 98 | 3.04% |
| Oxidase | | | 5.00% | 77 | |
| | | | 1.00% | 7 | |

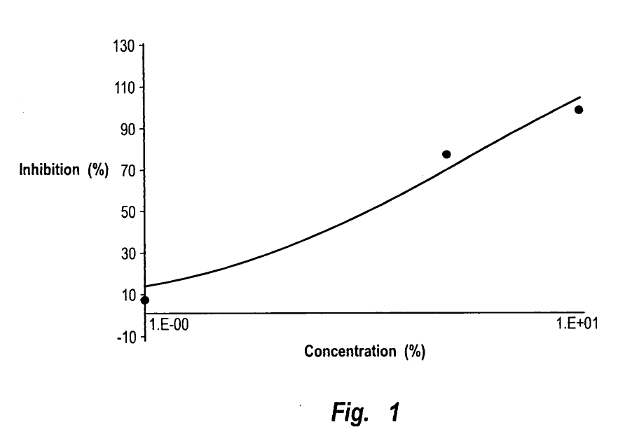

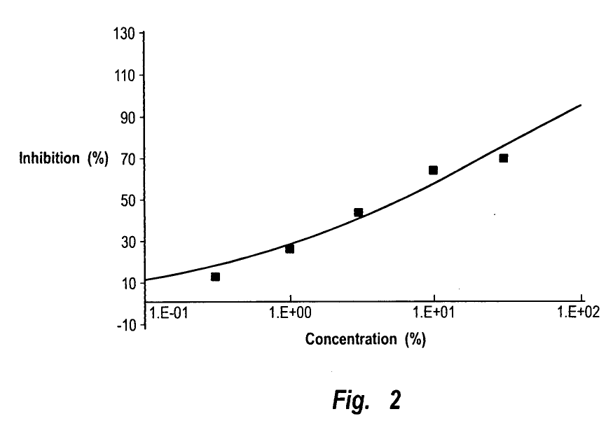

Table 3 represents a reference data set. Table 3 indicates that a concentration of 5 percent processed Morinda citrifolia juice results in 77 percent inhibition. As can be seen in Table 5, concentrations of 5 percent resulted in 77 percent inhibition, and that concentrations of 10 percent resulted in 98 percent inhibition. The data present in TABLE 4 is depicted in FIG. 1 as an inhibition curve. The inhibition curve set by diluted Morinda citrifolia can be compared with the inhibition curve of FIG. 2. FIG. 2 depicts the inhibition curve for Allopurinol. The inhibition curve for Allopurinol was produced utilizing the same procedures previously outlined for determining the inhibition curve for diluted Morinda citrifolia. A comparison of the inhibition curve for processed Morinda citrifolia (FIG. 1) products compared with the inhibition curve for Allopurinol (FIG. 2) illustrates the fact that processed Morinda citrifolia has substantial ability to inhibit xanthine oxidase.

### EXAMPLE FIVE

Additional assays were performed, which demonstrate the relationship percent inhibition of xanthine oxidase and concentration of processed Morinda citrifolia. The protocols described previously were utilized to produce the following data.

TABLE 5

| | | | | +% | | | | |
|---|---|---|---|---|---|---|---|---|
| | | | | INHIBITION | | | | |
| | | | | 100 | | | | |
| | | | | % | −100 | −50 | 0 | 50 |
| TARGET | SPP. | n= | CONC. | ↓ | ↓ | ↓ | ↓ | ↓ |

| | | | | |
|---|---|---|---|---|
| Xanthine | bov | 2 | 10% | 140 |
| Oxidase | | 2 | 5% | 113 |
| | | 2 | 1% | 11 |

As can be seen in Table 5, concentrations of 5 percent result in over 100 percent inhibition, and it appears that concentration of less then 5 percent will also result in significant inhibition.

Because hyperuricemia is found in approximately 25 percent of patients with osteoarthritis and 60 percent of patients with gout arthritis, XO inhibition can have a great impact on these patients.

Because the prevalence of hyperuricemia and gout increases with age as does the incidence of adverse affects of Allopurinol, aged patients are currently left without an effective medication for gout that does not present significant side effects. It has been suggested that the age-dependent acceptance or efficiency of Allopurinol is because of the age-dependent decline in renal function.

In addition to its uricosuric action, oxypurinol has a xanthuric affect which is also diminished in the elderly. Because of the increased efficacy of the Morinda Citrifolia treatment, even those elderly patients experiencing gout can be treated without the inherent side effects associated with the administration of Allopurinol.

As a result, there has been a long-felt need for a medication for the elderly to treat gout which does not have offsetting deleterious side effects. Further fractionation work with Morinda Citrifolia juice may reveal the active fraction and allow even further concentration of the effective fraction.

### EXAMPLE SIX

In this study, the selectivity of COX-2 inhibition of processed Morinda citrifolia products versus COX-1 in vitro was investigated. The inhibitions of processed Morinda citrifolia products on COX-2 and COX-1 activities were compared with that of the traditional NSAIDs such as Aspirin, Indomethacin, and a known selective COX-2 inhibitor, Celebrex. The COX-1 and COX-2 activities were determined based upon the PGE2 levels generated during the incubations of human platelets with tested compounds and/or vehicle by the Amersham ELA assay. The IC of processed Morinda citrifolia products, Aspirin, Indomethacin, and Celebrex on COX-1 are 5%, 4.55 μmol/L, 0.01 μmol/L, and 1.4 μmol/L, respectively, and that for COX-2 are 3.8%, 595 μmol/L, 0.4 μmol/L, and 0.47 μmol/L respectively. The data was converted into a ratio of IC₅₀ COX-2/COX-1. It was 0.76 for processed Morinda citrifolia products, 119 for Aspirin, 40 for Indomethacin, and 0.34 for Celebrex. These results show that the selectivity of COX-2 inhibition of processed Morinda citrifolia products is comparable with that of Celebrex. The discovery of the selective COX-2 inhibition of processed Morinda citrifolia products is very significant since processed Morinda citrifolia products is a natural fruit juice without side effects. This is the first scientific evidence for a strong anti-inflammatory activity in processed Morinda citrifolia products, which may also be one mechanism of treating the symptoms associated with gout.

While illustrative embodiments of the invention have been described herein, the present invention is not limited to the various preferred embodiments described herein, but includes any and all embodiments having modifications, omissions, combinations (e.g., of aspects across various embodiments), adaptations and/or alterations as would be appreciated by those in the art based on the present disclosure. Consequently, the present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope. The limitations in the claims are to be interpreted broadly based the language employed in the claims and not limited to examples described in the present specification or during the prosecution of the application, which examples are to be construed as non-exclusive. For example, in the present disclosure, the term “preferably” is non-exclusive and means “preferably, but not limited to.”

I claim:

1. A method for inhibiting xanthine oxidase, comprising:
administering a processed Morinda citrifolia product comprising:
Morinda citrifolia fruit juice present between about 50 and 99.9 percent by weight; and
another fruit juice present between about 0.1 and 50 percent by weight.

2. The method of claim 1, wherein said processed Morinda citrifolia product is further comprised an ingredient selected from a list consisting of: Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate.

3. The method of claim 1, wherein said Morinda citrifolia product is used with a carrier medium.

5. The method of claim 1, wherein said Morinda citrifolia product further comprises Quercetin present in an amount between 0.1 and 90 percent by weight.

6. The method of claim 5, wherein said processed Morinda citrifolia product further comprises Rutin present in an amount between 0.1 and 90 percent by weight.

7. The method of claim 1, wherein the other fruit juice is selected from a list consisting of: apple juice and blueberry juice.

8. A method for inhibiting xanthine oxidase, comprising the steps of:
adding a Morinda citrifolia product to solution selected from a list comprised of: water, hot water, alcohol, and an organic solvent;
isolating and extracting an active ingredient of said processed Morinda citrifolia product from said solution to obtain a fraction;
introducing said extracted active ingredient into or onto said mammal, wherein said extracted active ingredient inhibits xanthine oxidase.

9. A composition for inhibiting xanthine oxidase, comprising:
Morinda citrifolia fruit juice present between about 50 and 99.9 percent by weight; and
another fruit juice present between about 0.1 and 50 percent by weight.

10. The composition of claim 9, further comprising an ingredient selected from a list consisting of: Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate.

11. The composition of claim 10, wherein said Morinda citrifolia product is used with a carrier medium.

12. The composition of claim 9, wherein said composition is administered by process selected from a list consisting of: orally, transdermally, injection, intravenously, topically and systemically.

13. The composition of claim 9, further comprising the active ingredient Quercetin.

14. The composition of claim 13, further comprising Rutin.

15. The method of claim 1, wherein the other fruit juice is selected from a list consisting of: apple juice and blueberry juice.
